(19) United States
(12) Patent Application Publication
(10) Pub. No.: US 2007/0260078 A1
(43) Pub. Date: Nov. 8, 2007

(54) Integrated process for the manufacture of biodiesel

(76) Inventors: Ramanath N. Bhat, Billerica, MA (US); Nayantara Bhat, Billerica, MA (US)

Correspondence Address: VANCE INTELLECTUAL PROPERTY, PC, 5467 HILL TOP STREET, CROZET, VA, 22932-3167, US

(21) Appl. No.: 11/418,853

(22) Filed: May 5, 2006

(51) Int. Cl. C07C 51/43

(52) U.S. Cl. 554/174

(57) ABSTRACT

The present application describes an integrated process for the manufacture of biodiesel from vegetable or animal oils using a solid, transesterification catalyst and a solid, etherification catalyst.

### FIELD OF THE INVENTION

The present invention relates to an integrated process for the manufacture of biodiesel from vegetable or animal oils. More particularly, it relates to a process for the manufacture of biodiesel by reacting, in a cascade operation, the fatty acid triglycerides in vegetable or animal oils with an alcohol, first over a solid, transesterification catalyst and, subsequently, over a solid, etherification catalyst to form a mixture of alkyl esters of fatty acids and alkyl ethers of glycerol.

### BACKGROUND

Biodiesel is a liquid fuel obtained from biological and renewable sources like vegetable or animal oils and having physical, thermal, and other fuel characteristics similar to conventional diesel fuel obtained from crude petroleum oil. Biodiesels are expected to be important components of the alternate fuels that are needed to replace the currently used petroleum-derived diesel fuels, hereinafter referred to as petrodiesel. Examples of vegetable and animal oils from which biodiesel can be obtained are soyabean oil, rapeseed oil, corn oil, sunflower oil, cotton seed oil, jojoba oil, palm oil, olive oil, coconut oil, and margarine oil. The major constituents of such vegetable or animal oils are the triglyceride esters of fatty acids like oleic acid, stearic acid, and palmitic acid. In addition to the triglycerides, free, unesterified, fatty acids are also present in amounts even up to 20% in some cases. Because of their higher boiling points, viscosities, pour points, cloud points, and poorer cold flow properties, vegetable oils cannot be used directly, without any chemical transformation, as a substitute for petrodiesel.

When the fatty acid triglycerides in the vegetable or animal oil are transesterified with an alcohol like methanol or ethanol, the resulting methyl or ethyl esters of the fatty acids have physical characteristics similar to petrodiesel and can, hence, be used as a substitute for petrodiesel. In addition to the methyl or ethyl esters of the fatty acids, the products of transesterification also include glycerol to the extent of about 10% by wt.

Complete conversion of the triglyceride involves three consecutive reactions with monoglyceride and diglyceride intermediates:
Triglycerides+CH₃OH ⇄diglycerides+R₁COOCH₃
Diglycerides+CH₃OH ⇄monoglycerides+R₂COOCH₃
Monoglycerides+CH₃OH ⇄glycerol+R₃COOCH₃
where the R₁, R₂ and R₃ are the fatty acid alkyl groups. The mixture of R₁COOCH₃, R₂COOCH₃ and R₃COOCH₃ constitute the “biodiesel”. Conventional, commercial samples of biodiesel may also contain small quantities of unconverted mono- and diglycerides as well as free fatty acids.

Processes and catalysts for the transesterification of fatty acid triglycerides in vegetable oils with alcohols to alkyl esters of fatty acids and glycerol are well known in the prior art. These are (1) processes using homogeneous, basic catalysts like NaOH, KOH, alkali and alkaline metal hydroxides, alcoholates, acetates, and carbonates; (2) enzymatic transformations using enzymes like lipase; and, (3) processes using solid catalysts like calcium aluminate and zinc aluminate.

European Patent Application No. 0523767 describes a process, which involves the continuous use of a basic homogeneous catalyst, like sodium hydroxide. For homogeneous catalysts, high conversions are easy to achieve at low temperatures, in the range of 40 to 65° C. and a few hours of reaction; higher temperatures are not used to avoid system pressures greater than atmospheric that require the use of pressure vessels. These types of processes, however, have a number of disadvantages. Once the reaction is over, the excess catalyst that is essentially present in the glycerol phase in the form of alcoholates and sodium salts of fatty acids (“soaps”) must be removed after neutralization with an acid. Next, the water and alcohol must be removed by evaporation. The alcohol that evaporates usually has to be distilled. Traces of alkaline compounds are removed from the ester fraction by washing with water and drying. In addition, to get a glycerol fraction of sufficient purity for commercial use, a chain of complex and laborious treatments must be done thereby increasing the cost of production of glycerol. A major drawback of homogeneous basic catalysts like NaOH, KOH, Na₂CO₃, Na— and K— alcoholates, is that they are not effective when the vegetable oil contains significant fractions, say, above 5% by wt, of free fatty acids. These free fatty acids combine with the base catalyst forming sodium and potassium salts of the fatty acids (“soaps”). Since many natural vegetable and animal oils and especially all used or waste vegetable or animal oils contain free fatty acids to an extent greater than 5% by wt., homogeneous, base catalysts cannot be used in any economical manufacture of biodiesel from vegetable or animal oils.

U.S. Pat. Nos. 6,855,838; 6,822,105; 6,768,015; 6,712,867; 6,642,399; 6,399,800; 6,398,800; 6,398,707; 6,015,440; 5,578,090; and, 5,713,965 describe the use of enzyme catalysts like lipase in the transesterification of vegetable oils to alkyl esters of fatty acids and glycerol. The high cost of the lipase enzyme production is a major barrier to the commercialization of such enzymatic processes.

U.S. Pat. No. 6,878,837 describes a process for producing alkyl esters of fatty acids and glycerin employing a heterogeneous catalyst based on zinc aluminate. In this process, the water content in the reactor medium is controlled to a value that is below a given limiting value. U.S. Patent application US20020035282A1 describes a process for the alcoholysis of fatty acid glycerides in the presence of a catalyst containing an alkali metal, alkaline earth metal, or zinc carbonates at 160-300° C. One significant drawback in this process is that the alkali metal, alkaline earth metal, or zinc carbonates used as catalysts in this process have significant solubility in the alcohols used in the transesterification reaction. Hence, they have to be separated and recovered from the effluents of the transesterification reaction to obtain the pure biodiesel. In fact, Example 1 of this patent application specifically mentions that the biodiesel-containing product was washed four times with 1 N HCl to remove the carbonate catalyst. It is desirable to discover a solid catalyst that is not at all or only negligibly soluble in the reaction medium during the transesterification reaction.

U.S. Pat. No. 5,508,457 describes the transesterification of vegetable oils over solid catalysts, ETS-4 and ETS-10. Conversions of 85.7 and 52.7%, respectively, were obtained. Other solid catalysts like Cs-exchanged NaX zeolite and hydrotalcite have also been reported in the transesterification of rapeseed oil by Leclerque et al in the J. A. Oil Chem. Soc. 2001, 78, 1161. At a high methanol to oil ratio of 275 and 22 hours of reaction time at methanol reflux, the Cs—NaX zeolite gave a conversion of 70% by wt.; the conversion over hydrotalcite was 34%. More recently, Suppes et al in the J. Appl. Cat. 2004, 257, 213 reported the transesterification of Soya bean oil with methanol over zeolites, titanosilicates, and metal catalysts. They found that the titanosilicate, ETS-10, was the best converting 92% of the oil to biodiesel after 24 hours of reaction at 120° C. They also observed that a major drawback of transesterification processes using zeolites and other such oxides with ion exchange properties is that during the reaction, significant leaching of the metal cations occurs from the solid catalyst into the reaction solution. As a consequence of such leaching, the catalyst cannot be reused or used continuously over a long period of time, thereby increasing the amount of catalyst consumed. The free fatty acids present in the vegetable or animal oil formed the metal salts of the carboxylic acids (“soap”) poisoning the catalyst activity. Their data indicated that free fatty acids inhibit solid catalysts that rely only on highly basic sites.

A major limitation in most of the prior art processes for the manufacture of biodiesel is the absence of a convenient method for the conversion of glycerol, a byproduct of the transesterification process and which is produced in yields of about 10% by wt of the vegetable oil, into a large volume, blending component of the biodiesel. While a large number of processes for converting glycerol into important, but relatively small volume chemicals like propanediol and others are available, such applications cannot provide a commercial outlet for the very large, fuel-like volumes of glycerol that will be produced as a byproduct once large volumes of biodiesel are manufactured.

Ideally, it is desirable to convert glycerol into a fuel component that can be compatibly blended with the alkyl esters of the fatty acids to constitute the biodiesel. One such process is described in U.S. Pat. Nos. 6,174,501 and 6,015,440. These two patents together claim a process for producing biodiesel wherein the triglycerides are reacted in a liquid phase reaction with a homogeneous basic catalyst, like NaOH, and an alcohol such as ethanol or methanol. The crude glycerol obtained as a product is first separated from the major product, the alkyl esters of fatty acids, flashed to remove excess alcohol, passed through strong cationic ion exchangers to remove anions, and is then reacted with isobutylene or isoamylene in the presence of a strong acid catalyst to produce glycerol ethers. The glycerol ethers are then added back to the transesterified triglycerides to provide the biodiesel. The alkyl ethers of glycerol are completely miscible with the alkyl esters of fatty acids in the liquid phase in all proportions. A mixture comprising alkyl esters of fatty acids and alkyl ethers of glycerol has all the physical and functional properties, like density, viscosity, kinematic viscosity, pour point, cloud point, lubricity, and calorific value that are characteristic of biodiesel. The process described in U.S. Pat. Nos. 6,174,501 and 6,015,440 has the advantage that it increases the volumetric amount of biodiesel that can be obtained from a given volume of vegetable or animal oil in that it converts the byproduct of the transesterification reaction, glycerol, into a compound that can be blended with the methyl esters of the fatty acids. But the process described in U.S. Pat. Nos. 6,174,501 and 6,015,440 also suffers from the limitation that a large amount of energy is expended in the separation of the methyl esters of fatty acids from the byproduct, crude glycerol, and the further removal of alcohol and water from the glycerol fraction and additional purification of glycerol by passage through strong cationic ion exchangers to remove the anionic impurities. This laborious and expensive procedure for purification of glycerol is necessary since the above-mentioned U.S. Pat. Nos. 6,174,501 and 6,015,440 etherify the glycerol with olefins, like isobutylene, over strong acids and such olefins cannot be used as etherifying agents of glycerol if impurities like water, alcohol, or anionic species, such as hydroxide ions, are also present in the reaction mixture. Anionic impurities arise when homogeneous catalysts, like sodium hydroxide, are used for the transesterification reaction, as in the process claimed in U.S. Pat. Nos. 6,174,501 and 6,015,440. Another limitation of the process of U.S. Pat. Nos. 6,174,501 and 6,015,440 is that olefins like isobutylene and isoamylene are used for the etherification of glycerol. These olefins are available in refineries processing crude petroleum oils and are not normally available in biorefineries wherein the vegetable and animal oils, the raw material for the biodiesel, are utilized. While this process increases the overall yield of biodiesel from a given amount of vegetable oil by converting the byproduct glycerol to a diesel blending component, namely, alkyl ethers of glycerol, the high energy consumption involved in the isolation and purification of glycerol for the subsequent etherification reaction reduces its economic profitability. Claim 1 of U.S. Pat. No. 6,174,501, for example, specifically invokes the use of a triglycerides/crude glycerol separator unit as well as a flash unit for separating the crude glycerol and alcohol.

An additional limitation of the process of U.S. Pat. Nos. 6,174,501 and 6,015,440 is that since they use homogeneous basic catalysts like NaOH, they cannot operate successfully when the oil contains free fatty acids since cations like Na, K, Ca, and Zn when present in a homogeneous, liquid phase, form soaps, which are metal salts of the free fatty acids. The formation of soaps during the transesterification reaction renders the process of separation of the biodiesel from the soap too cumbersome and uneconomic.

A process improvement wherein (1) solid catalysts are used in both the transesterification and etherification reactions, (2) the effluents from the transesterification stage can be used directly, without further separation and purification of the crude glycerol, in a cascade operation for the etherification of glycerol, and (3) alcohols, instead of olefins can be used for the etherification reaction would be a significant advance over current processing.

In view of the above, there is a need for an improved and integrated process for the manufacture of biodiesel which (1) uses solid catalysts for both the transesterification of the vegetable or animal oil and etherification of glycerol, (2) does not involve the separation of the glycerol from the methyl esters of fatty acids prior to the etherification reaction and (3) which can use alcohols as etherifying agents for glycerol.

### SUMMARY OF THE INVENTION

In an aspect, the present invention provides a process for the manufacture of biodiesel from vegetable or animal oils by transesterifying the oil in a transesterification reactor and further reacting the transesterification products in an etherification reactor to provide biodiesel.

This and other aspects, which will become apparent during the following detailed description, have been achieved by the inventor's discovery that the presently claimed process and catalysts are useful for manufacturing biodiesel.

### DETAILED DESCRIPTION OF PREFERRED EMBODIMENTS

Accordingly, in an embodiment, the present invention provides a process for the manufacture of biodiesel comprising:
(a) transesterifying, in a first reactor zone, an oil with an alcohol in the presence of a solid transesterification catalyst to form a mixture of glycerol and alkyl esters of fatty acids; and,
(b) contacting the effluents from the first reactor zone, with a solid, acidic, etherification catalyst in a second reactor zone to form glycerol ethers;
   wherein the oil is selected from a vegetable oil, an animal oil, or a mixture thereof.

Each reactor zone has a feed or starting material as well as an effluent. Feed means the materials that are delivered to the reactor zone (i.e., the starting materials for the reactor zone). For example, in the first reactor zone it comprises the starting mixture of alcohol and oil which is to be reacted. In the second reactor zone, it comprises the effluents from the first reactor zone, unless they are modified (e.g., the alkyl esters of fatty acids are at least partially removed).

Typically, the alcohol used in the first reactor zone is carried through in the effluents from the first reactor zone into the second reactor zone for use in the etherification process. Additional alcohol can be added to the second reactor zone, if desired. Alternatively, a second (different) alcohol can be added to the second reactor zone. Alternatively, two or more different alcohols can be used in the first and/or second reactor zone if desired. The present process can be advantageously run via a cascade operation without separation and/or purification of the resulting alkyl esters and/or the resulting glycerol. By converting the glycerol component into glycerol ethers, the yield of biodiesel can be enhanced over procedures not making such a conversion.

In another embodiment, the process further comprises: (c) removing alcohol present in the effluents of the second reactor zone. Typically, the present process is run in the presence of an excess of alcohol (e.g., the moles of alcohol are greater than the total moles of fatty acids and glycerol). This alcohol can be removed by methods known to those of ordinary skill in the art (e.g., distillation).

In another embodiment, the process further comprises: (d) recycling the removed alcohol back to the transesterification step.

In another embodiment, the solid transesterification catalyst is a double metal cyanide catalyst of the formula M₂M¹₃(CN)₁₀, wherein M and M¹ are independently metals having oxidation states of 2 or 3. Examples of M and M¹ independently include iron, cobalt, zinc, and nickel. Examples of catalysts include (a) Fe₂Zn₃(CN)₁₀, Co₂Zn₃(CN)₁₀, Ni₂Zn₃(CN)₁₀, Fe₂Co₃(CN)₁₀, Fe₂Ni₃(CN)₁₀, Co₂Ni₃(CN)₁₀, and Fe₂Fe₃(CN)₁₀ and (b) Fe₂Zn₃(CN)₁₀, Co₂Zn₃(CN)₁₀, and Ni₂Zn₃(CN)₁₀.

Vegetable and animal oils are triglyceride-containing oils. In another embodiment, the vegetable oil is selected from soyabean oil, rapeseed oil, corn oil, sunflower oil, cottonseed oil, jojoba oil, palm oil, olive oil, coconut oil, margarine oil, rubberseed oil, and mixtures thereof. Examples of oils include used cooking oils (e.g., vegetable or animal) comprising at least 1% by wt of free fatty acids. Examples of used oils include corn, soyabean, sunflower oil, rubberseed oil, palm oil, rapeseed oil, linseed oil, peanut oil, canola oil, cottonseed oil, tallow, lard, yellow grease, and mixtures thereof.

In another embodiment, the molar ratio of alcohol to triglyceride in the oils in the feed to the first reactor zone is greater than 6. It is generally desirable for the amount of alcohol present in the feed or starting materials of the first reactor zone to be sufficient for not only the esterification reaction, but also the etherification reaction.

In another embodiment, the alcohol in the feed to the first reactor zone is a C₁₋₁₀ alcohol. C₁₋₁₀ alcohols include both linear and branched alkyl chains that are terminated with a hydroxy group. Examples of alcohols include (a) methanol, ethanol, propanol, isopropyl alcohol, n-butanol, s-butanol, and t-butanol; and, (b) methanol and ethanol.

In another embodiment, when free fatty acids are present in the oil (e.g., vegetable oil) being transesterified, the free fatty acids are converted to alkyl esters of fatty acids over the solid, double metal cyanide catalysts.

In another embodiment, the temperature of the reaction in the first reactor zone is from 60, 70, 80, 90, 100, 110, 120, 130, 140, 150, 160, 170, 180, 190, 200, to 200° C.

In another embodiment, the pressure in the first reactor zone is from 1, 2, 3, 4, 5, 10, 15, 20, 25, 30, 35, 40, 45, to 50 bar.

In another embodiment, the pressure in the first reactor is kept at the autogenous pressure of the alcohol at the transesterification reaction temperature.

In another embodiment, the solid, acidic, etherification catalyst in the second reactor is selected from zeolite beta, zeolite Y, and an acidic, polymeric cation exchange resin. Examples of solid, acidic, etherification catalysts include Amberlyst-15 (e.g., Aldrich Co., USA) and an aluminosilicate zeolite (e.g., zeolite beta). The zeolite catalysts are well known and commercially available from companies such as Zeolist Corporation (USA), Sud-Chemie (Germany), and TOSO (Japan).

In another embodiment, the temperature of the reaction in the second reactor zone is from 120, 130, 140, 150, 160, 170, 180, 190, to 200° C.

In another embodiment, the pressure in the second reactor zone is below 30 bar. Examples of the pressure in the second reactor zone include 10, 15, 20, 25, to 30 bar.

In another embodiment, the pressure in the second reactor zone is kept at the autogenous pressure of the alcohol at the etherification reaction temperature. As noted above, the alcohol in the second reactor zone is typically the alcohol from the first reactor zone.

In another embodiment, the alkyl esters of fatty acids are partially removed from the effluent from the first reactor zone before contacting the effluents with the solid etherification catalyst in the second reactor zone. These esters can be removed by techniques known to those of skill in the art (e.g., distillation). The separated alkyl esters can be mixed with the glyceryl ethers from the second reactor zone. For example, the separated alkyl esters can be blended with the effluents from the second reactor zone after the excess alcohol has been removed.

The present invention may be embodied in other specific forms without departing from the spirit or essential attributes thereof. This invention encompasses all combinations of aspects of the invention noted herein. It is understood that any and all embodiments of the present invention may be taken in conjunction with any other embodiment or embodiments to describe additional embodiments. It is also to be understood that each individual element of the embodiments is intended to be taken individually as its own independent embodiment. Furthermore, any element of an embodiment is meant to be combined with any and all other elements from any embodiment to describe an additional embodiment.

The process of the present invention can be carried out in different reactor configurations. Examples of configurations include: (1) two continuous stirred tank reactors connected in series; (2) two fixed bed, plug flow reactors connected in series; (3) a continuous stirred tank first reactor connected in series with a fixed bed, plug flow second reactor; and, (4) a fixed bed, plug flow first reactor connected in series with a continuous stirred tank second reactor.

The present invention has a number of potential advantages over current processes for the manufacture of biodiesel. These advantages can include:
a. The present invention can maximize the volume yield of biodiesel from a given quantity of the vegetable and/or animal oil by converting the glycerol fraction, a byproduct during the transesterification reaction, to glyceryl ethers of alcohols. The glyceryl ethers can be blended without any miscibility problems with the alkyl esters of fatty acids thereby increasing the net yield of the biodiesel from the process. The cetane number of the biodiesel can be improved by the presence of the alkyl glyceryl ethers formed in the second reactor. The alkyl glyceryl ethers introduce oxygenates in the biodiesel thereby contributing to the more complete combustion of the fatty acid alkyl esters. Current processes for the manufacture of biodiesel generally do not provide for the conversion of glycerol into a biodiesel component.
b. The present invention can avoid elaborate and expensive separation of the alkyl esters of fatty acids from the effluents of the transesterification reactor before etherifying the residual byproduct, glycerol, to glyceryl ethers (if such etherification is even conducted).
c. The present invention utilizes solid catalysts that are not soluble in the reaction mixture during the transesterification or etherification reactions. These non-soluble catalysts can allow the present invention to avoid the use of elaborate and expensive purification procedures to recover the glycerol for the etherification reactions.
d. With respect to conventional homogeneous basic catalysts, the present solid catalysts generally do not require numerous steps for purifying the products formed, which contain all of the homogeneous basic catalyst. Thus, waste and their processing can be avoided. No polluted effluent needs to be discharged.
e. The glycerol produced by present process is generally free of salts and is, usually, at least 98% pure.

When using fixed bed processes based on solid catalysts in the present invention a number of advantages can be obtained. They can include:
a. Substantial reduction of waste/byproduct generation, savings on catalyst costs;
b. Use of recuperative heating reduces heat requirements as compared to batch processes;
c. Considerably greater increase in reactor throughput;
d. Smaller heat exchanger areas (condensers, heat addition) and corresponding reduced costs; and,
e. Greater use of automation and continuous processing without operator on duty.

The solid double metal cyanide catalyst used in the present process are known for other uses and have been utilized for the commercial production of polyether glycols and other chemical products. U.S. Pat. No. 5,789,626, for example, describes the preparation and application of one member of this group of double metal cyanide catalysts in the manufacture of high quality polypropylene glycol products that have a low level of unsaturation, narrow molecular weight distributions, and low viscosity. Kim et al (Polymer 2003, 44, 3417) describe a method for preparing this type of catalyst and have demonstrated their high activity for ring opening polymerization of propylene oxide.

The solid double metal cyanide catalysts are conveniently prepared by reacting an aqueous solution of ZnCl₂, an aqueous solution of K₄Fe(CN)₆, and a polymer solution. Examples of commercially available polymer solutions that can be used in the preparation are polypropylene glycol, polyethylene glycol, and solutions of copolymers of polyethylene glycol and polypropylene glycol (e.g., triblock, poly(ethylene glycol)-block-poly(propylene glycol)-block-poly(ethylene glycol)(EO₂₀-PO₇₀-E₂₀)) of molecular weights in the region of 4000 to 7000. The solid double metal cyanide catalysts are insoluble in almost all solvents including aqua regia. The insolubility of these solid catalysts in almost all solvents has a major advantage in liquid phase reactions since the possibility of the metal ions leaching out during the reactions is negligible. Such leached ions, apart from causing environmental problems in the disposal of the reaction effluents, render the isolation and purification of the desired reaction products more difficult and expensive.

All examples provided above are not intended to be limiting unless indicated.

Other features of the invention will become apparent in the course of the following descriptions of exemplary embodiments that are given for illustration of the invention and are not intended to be limiting thereof.

### EXAMPLES

### Example 1

K₄[Fe(CN)₆] (0.02 moles) was dissolved in a mixture of water (80 mL). ZnCl₂ (0.2 moles) was dissolved in a mixture of water (200 mL) and tertiary butanol (40 mL). Triblock, poly(ethylene glycol)-block-poly(propylene glycol)-block-poly(ethylene glycol)(EO₂₀-PO₇₀-E₂₀), of molecular weights 5800 (30 g) was dissolved in a mixture of tertiary butyl alcohol (80 mL) and of water (4 mL). The zinc-containing solution was added to the Fe-containing solution over a period of 60 minutes at 50° C. with vigorous stirring. A white precipitate was formed. The polymer solution was then added over a period of 5 minutes. Stirring was continued for a further 12 hours. The solid cake that was formed was filtered, washed with distilled water, and dried in air at 25° C. for 2 days. The material was heated in air at 180° C. for 4 hours. The resulting catalyst had a chemical composition Fe₂Zn₃(CN)₁₀.

### Example 2

Sunflower oil (5 g) and ethanol (ethanol to oil molar ratio of 10:1) were preheated to 150° C. in a preheater and passed through a first reactor zone consisting of a fixed bed catalyst containing the solid catalyst (1 g) prepared in Example 1, at a weight hourly space velocity of 0.25 gram of oil per gram of solid catalyst per hour. The catalyst was maintained at 170° C. The pressure in the first reactor was maintained at 10 bar. The effluents from the first reactor, consisting mainly of glycerol, ethyl esters of fatty acids and unreacted ethanol were cooled to 90° C. and passed through a second reactor zone containing an acidic, polymeric cation exchange resin (1 g), Amberlyst-15 at a weight hourly space velocity of 0.25 gram of the total effluent per gram of solid catalyst per hour. The polymeric cation exchange resin was maintained at 90° C. The pressure in the second reactor was maintained at 1 bar. The effluents from the second reactor, containing ethyl esters of fatty acids, glycerol triethyl ether, and unreacted alcohol, were cooled to room temperature in a condenser cooled by water. The unreacted alcohol was removed from the product by flash distillation leaving behind the mixture of ethyl esters of the fatty acids and glycerol triethyl ether. This mixture of ethyl esters of the fatty acids and glycerol triethyl ether constitutes a biodiesel. Mass balance and gas chromatographic analysis of the product revealed that the conversion of the sunflower oil was almost 100%. The molar yields of ethyl esters as well as the triethyl glyceryl ether were higher than 95%. The density of the biodiesel at 15° C. was 0.82 gm/cc. Its pour point was −10° C. Its kinematic viscosity at 40° C. was 5.6 CST.

### Example 3

Example 2 was repeated except that instead of ethanol, methanol was used as the alcohol. Mass balance and gas chromatographic analysis of the product revealed that the conversion of the sunflower oil was almost 100%. The molar yields of methyl esters as well as the trimethyl glyceryl ether were higher than 90%. The density of the biodiesel at 15° C. was 0.81 gm/cc. Its pour point was −12° C. Its kinematic viscosity at 40° C. was 5.1 CST.

### Example 4

Example 1 was repeated except that, instead of potassium ferrocyanide, K₄[Fe(CN)₆], the cobalt analog, potassium cobaltocyanide, K₄[Co(CN)₆], was used. The resulting catalyst was Co₂Zn₃(CN)₁₀.

### Example 5

Example 1 was repeated except that, instead of potassium ferrocyanide, K₄[Fe(CN)₆], the nickel analog, potassium nickel cyanide, K₄[Ni(CN)₆], was used. The resulting catalyst was Ni₂Zn₃(CN)₁₀.

### Example 6

Example 2 was repeated except that instead of the catalyst of Example 1, the catalyst of Example 4 was used as the transesterification catalyst in the first reactor. Amberlyst-15 was used, as in Example 2, as the etherification catalyst. Mass balance and gas chromatographic analysis of the product revealed that the conversion of the sunflower oil was 95%. The molar yields of ethyl esters as well as the triethyl glyceryl ether were above 90%. The density of the biodiesel at 15° C. was 0.82 gm/cc. Its pour point was −11° C. Its kinematic viscosity at 40° C. was 5.6 CST.

### Example 7

Example 2 was repeated except that instead of the catalyst of Example 1, the catalyst of Example 5 was used as the transesterification catalyst in the first reactor. Amberlyst-15 was used, as in Example 2, as the etherification catalyst. Mass balance and gas chromatographic analysis of the product revealed that the conversion of the sunflower oil was 95%. The molar yields of ethyl esters as well as the triethyl glyceryl ether were above 90%. The density of the biodiesel at 15° C. was 0.81 gm/cc. Its pour point was −10° C. Its kinematic viscosity at 40° C. was 5.6 CST.

### Example 8

Example 2 was repeated except that instead of the polymeric cation exchange resin, Amberlyst-15, a commercial sample of an aluminosilicate zeolite, H-beta of silica to alumina ratio=40, was used as the etherification catalyst in the second reactor. Mass balance and gas chromatographic analysis of the product revealed that the conversion of the sunflower oil was 100%. The molar yields of ethyl esters as well as the triethyl glyceryl ether were above 96%. The density of the biodiesel at 15° C. was 0.81 gm/cc. Its pour point was −10° C. Its kinematic viscosity at 40° C. was 5.6 CST.

### Example 9

Example 2 was repeated except that instead of sunflower oil, other oils like rapeseed oil, corn oil, sunflower oil, cottonseed oil, jojoba oil, palm oil, olive oil, coconut oil, and margarine oil were used. In all cases excellent yields of the corresponding ethyl esters and triethyl glycerol ether, usually above 90%, were obtained.

### Example 10

Example 2 was repeated except that instead of fixed bed reactors, both the first and second reactors were continuous stirred tank reactors. The residence time in both the reactors was 5 hours each. The conversion of the oil was almost 100%. The molar yields of the alkyl esters and glyceryl ethers were also above 95%. The density of the biodiesel was 0.82 gm/cc. Its pour point was −12° C.

Numerous modifications and variations of the present invention are possible in light of the above teachings. It is therefore to be understood that within the scope of the appended claims, the invention may be practiced otherwise that as specifically described herein.

What is claimed is:

1. A process for the manufacture of biodiesel comprising:
(a) transesterifying, in a first reactor zone, an oil with an alcohol in the presence of a solid transesterification catalyst to form a mixture of glycerol and alkyl esters of fatty acids; and,
(b) contacting the effluents from the first reactor zone, with a solid, acidic, etherification catalyst in a second reactor zone to form glycerol ethers;
wherein the oil is selected from a vegetable oil, an animal oil, or a mixture thereof.

2. The process of claim 1, further comprising:
(c) removing alcohol present in the effluents of the second reactor zone.

3. The process of claim 2, further comprising:
(d) recycling the removed alcohol back to the transesterification reaction.

4. The process of claim 1, wherein solid transesterification catalyst is a double metal cyanide catalyst of the formula M₂M¹₃(CN)₁₀, wherein M and M¹ are independently metals having oxidation states selected from 2 and 3.

5. The process of claim 4, wherein M and M¹ are independently selected from iron, cobalt, zinc, and nickel.

6. The process of claim 5, wherein the catalyst is selected from Fe₂Zn₃(CN)₁₀, Co₂Zn₃(CN)₁₀, and Ni₂Zn₃(CN)₁₀.

7. The process of claim 1, wherein the oil is a vegetable oil and is selected from soyabean oil, rapeseed oil, corn oil, sunflower oil, cottonseed oil, jojoba oil, palm oil, olive oil, coconut oil, margarine oil, rubberseed oil, and mixtures thereof.

8. The process of claim 1, wherein the oil is a used oil selected from corn oil, soyabean oil, sunflower oil, rubberseed oil, palm oil, rapeseed oil, linseed oil, peanut oil, canola oil, cotton seed oil, tallow, lard, yellow grease, and mixtures thereof.

9. The process of claim 1, wherein the molar ratio of alcohol to triglyceride in the oil in the feed to the first reactor zone is greater than 6.

10. The process of claim 1, wherein the alcohol in the first reactor zone is a C₁₋₁₀ alcohol.

11. The process of claim 10, wherein the alcohol is selected from methanol and ethanol.

12. The process of claim 1, wherein when free fatty acids are present in the oil being transesterified, the free fatty acids are converted to alkyl esters of fatty acids over the transesterification catalyst.

13. The process of claim 1, wherein the temperature of the reaction in the first reactor zone is from 60-200° C.

14. The process of claim 1, wherein the pressure in the first reactor is kept at the autogenous pressure of the alcohol at the transesterification reaction temperature.

15. The process of claim 1, wherein the solid, acidic, etherification catalyst in the second reactor is selected from: zeolite beta; zeolite Y; and, an acidic, polymeric cation exchange resin.

16. The process of claim 15, wherein the solid, acidic, etherification catalyst is selected from Amberlyst-15 and an aluminosilicate zeolite.

17. The process of claim 1, wherein the temperature of the reaction in the second reactor zone is from 120-200° C.

18. The process of claim 1, wherein the pressure in the second reactor zone is kept at the autogenous pressure of the alcohol at the etherification reaction temperature.

19. The process of claim 1, wherein the process is carried out in a reactor configuration selected from: (1) two continuous stirred tank reactors connected in series; (2) two fixed bed. plug flow reactors connected in series; (3) a continuous stirred tank first reactor connected in series with a fixed bed, plug flow second reactor; and, (4) a fixed bed, plug flow first reactor connected in series with a continuous stirred tank second reactor.

20. The process of claim 1, wherein the process is run via a cascade operation without separation or purification of the effluents from the first reactor zone.

21. The process of claim 1, wherein the alkyl esters of fatty acids are removed from the effluents from the first reactor zone.

22. The process of claim 21, wherein the separated alkyl esters are mixed with the glyceryl ethers from the effluents of the second reactor zone.
